(19) United States
(12) Patent Application Publication
(10) Pub. No.: US 2024/0200015 A1
(43) Pub. Date: Jun. 20, 2024

(54) Movable Effector Assembly, Assembly, System, and Methods

(71) Applicant: NEOGEN Corporation, Lansing, MI (US)

(72) Inventors: Gregory W. Sitton, Lansing, MI (US); Daniel Crichton, Lansing, MI (US); Adrian M. Woodward, Lansing, MI (US); Simon A Shakespeare, Lansing, MI (US)

(21) Appl. No.: 18/251,540

(22) PCT Filed: Nov. 1, 2022
(86) PCT No.: PCT/IB2021/060099

§ 371 Date: Feb. 22, 2024

Related U.S. Application Data
(60) Provisional application No. 63/108,587, filed on Nov. 2, 2020.

(51) Int. Cl. C12M 1/36; B25J 13/00; C12M 1/00; C12M 1/12; C12M 1/26; C12M 3/00

(52) U.S. Cl. CPC C12M 41/48; B25J 13/00; C12M 23/48; C12M 23/50; C12M 25/04; C12M 33/04

(57) ABSTRACT

A moveable effector assembly designed for, among other things, moving culture devices, uncovering and replacing covers from culture devices. Culture device processing systems comprising such moveable effector assemblies. Methods of using such moveable effector assemblies and culture device processing systems.

### TECHNICAL FIELD

The invention relates to a system for processing culture devices. Specifically, a system for processing culture devices including a movable effector assembly

### BACKGROUND

The culturing microorganism samples is known and may be accomplished using a variety of culture devices, for example thin-film culture devices that are available under the PETRIFILM™ brand from 3M Company, St. Paul, MN, USA. Measurements or testing procedures, such as microorganism enumeration, can then be performed on a cultured sample.

The INOBOT, available from Tecnic (Dongen, Netherlands), is a robot capable of inoculating thin-film culture devices.

US20180164333 discloses a test plate processing system for processing multiple test plates.

### SUMMARY

This disclosure describes a movable effector assembly that has sufficient dexterity to manipulate culture devices, including thin-film culture devices, in order to perform at least some of the steps necessary to process the culture devices. The movable effector assemblies can be a component of the culture processing device; in this disclosure it is primarily discussed in that context. However, the moveable effector assemblies can be a component of other devices for manipulating and moving objects, such as an assembly line, a robotic or mechanical workstation, and the like. The effector assemblies described herein are referred to as being “movable” because they contain at least some movable components and because, in particular applications, they are positioned along a track and can move along the track.

A culture processing device can be configured to support or secure a culture device on a channel. In most cases, the channel provides a surface on which the culture device can rest, but it is also possible for the channel to include one or more securing devices, such as clips, straps, clamps, and the like, or even a vacuum securement system, for example, where the channel has small holes through which a vacuum can be applied. being operated, for example, by the various components of the culture processing device. A track, along which the moveable effector assembly can move, can be associated with the channel. By moving along the track, not only can the movable effector assembly manipulate the culture device, for example to lift, uncover, replace the cover on the culture device, stamp the inoculum over a growth area of the culture device, and apply a label to the culture device but it can also move the culture device between various position along the channel.

The movable effector assembly can have one or more effector arms. The one or more effector arms can include attachment members configured to engage and retain a culture device and to place the culture device on a location on a channel, for example an input location, an output location, or other locations, and to move the culture device between locations, for example from an input location to an output location. The effector arm is also capable of removing a cover from the culture device to expose the growth area for inoculation and stamping the culture device, particularly the growth area of the culture device, to spread the inoculant within the growth area. The effector arm is further capable of replacing the cover (typically after inoculation) over the growth area and transferring the culture device to an output location. The attachment members can be further configured to take a label from a labeler and place the label on the culture device.

The one or more effector arms can include a plurality of segments that are each rotatable around two or more parallel, non-colinear axes. The plurality of independently rotatable segments of the effector arm provides increased mobility and dexterity of the movable effector assembly. This allows the movable effector assembly to carry out a variety of actions or tasks with the precision required of actions or tasks that require fine motor skills, such as uncovering and replacing lids of culture devices, moving and process culture devices, as well as other actions or tasks that may require fine motor skills. When used in processing culture devices, the beneficial mobility and dexterity of the effector arm allows the movable effector assembly to move culture device between locations, to remove and replace the cover over the growth area, etc., in each case without jostling or shaking the culture device in a way that could spill the inoculant, interfere with incubation of microorganisms on the culture device, or damage the culture device.

The movable effector assembly of the culture device processing system can also include one or more stamping tools configured to press down on the culture device to spread the inoculant over the growth area. The one or more stamping tools can be customized or customizable for one or more different types of culture devices. The dexterity and mobility of the movable effector assembly permit it to spread of the inoculant over the growth area, a task that requires fine motor skills and that could previously be accomplished only manually and not mechanically or automatically.

The culture device processing system can also include an image capture device. The image capture device can capture an image of a culture device that is being processed, that is about to be processed, or that has been processed.

A computer or processer, which can be internal or external to the culture device processing system, can be in communication with the culture device processing system. The computer can execute a set of instructions that are recorded on a non-transient computer-readable storage media. When executed, the instructions can cause the culture processing device to process one or more culture devices. The non-transient computer-readable storage media can contain a plurality of sets of instructions, each set associated with a type of culture device. The computer or processer can receive, from the image capture device, one or more captured indicia relating to the type of culture device that is being processed and then retrieve, from the non-transient computer-readable storage media, one or more received indicia, and compare the one or more received indicia to the one or more captured indicia. This can decrease user error by ensuring that the correct set of instructions or processing protocol is performed on each culture device.

An inoculation system can contain a plurality of culture processing devices. The plurality of culture processing devices can be arranged to work independently or together to efficiently process a plurality of culture devices, thereby increasing throughput and decreasing user error.

### BRIEF DESCRIPTION OF DRAWINGS

FIG. 1 is a illustrates a perspective view of an embodiment of a culture device processing system;

FIG. 2 illustrates an isometric view of the embodiment of the culture device processing system including an effector assembly having a first effector arm and a second effector arm in a first position;

FIG. 3 illustrates an isometric view of the embodiment of the culture device processing system including the effector assembly having a first effector arm in a second position;

FIG. 4 illustrates a perspective view of a movable effector assembly;

FIG. 5 illustrates an isometric view of an inoculation system containing a plurality of culture device processing systems each coupled to a plurality of channels;

FIG. 6 illustrates the movable effector assembly coupled to a stamping tool;

FIG. 7 illustrates coupling of the first effector arm of the movable effector assembly to a culture device;

FIG. 8 illustrates the first effector arm placing of the culture device onto a first area of a channel;

FIG. 9 illustrates an image capture device of the processing apparatus capturing an image of the culture device;

FIG. 10 illustrates the first effector arm removing a cover of the culture device to expose a growth area of the culture device;

FIG. 11 illustrates the first effector arm covering the growth area of the culture device with the cover of the culture device;

FIG. 12 illustrates the stamping tool stamping the culture device;

FIG. 13A illustrates the first effector arm receiving and retaining a label from a labeler;

FIG. 13B illustrates a second effector arm receiving and retaining a label from a labeler;

FIG. 14A illustrates the first effector arm placing a label on the culture device;

FIG. 14B illustrates the second effector arm placing a label on the culture device;

FIG. 15 illustrates the first effector arm moving the culture device to an output location of the channel;

FIG. 16 illustrates the movable effector assembly returning to a location near the input location of the channel and releasing the stamping tool.

### DETAILED DESCRIPTION

Particular embodiments of the disclosure are shown in detail in the examples and Figures. However, the disclosure extends beyond the examples and Figures, so the scope of protection sought is to be limited only by the claims.

Certain terms are used in the following description for the purpose of reference only, and thus are not intended to by limiting. For example, terms such as “above” and “below” may refer to directions in the drawings to which reference is made, or to indicate that two elements are on the same or opposite side of a third element; however, they should not be understood to require any specific orientation unless otherwise specified. Terms such as “front,” “back,” “left,” “right,” “rear,” and “side” may describe the relative orientation, position, or location of components or elements within a consistent but arbitrary frame of reference to facilitate understanding of the text and Figures, but do not necessarily require any absolute orientation, position, or location. Moreover, terms such as “first,” second,” “third,” and so on may be used to describe separate components, but do not necessarily imply a chronological or special ordering.

The term “culture device” means and includes a device for culturing one or more microorganisms, and includes, for example, agar-based culture devices such as well as thin-film culture devices such as those sold under the PETRIFILM™ brand by from 3M Company, St. Paul, MN, USA

The term “longitudinal” refers to the direction extending between a first end of the culture device processing system and a second end of the culture device processing system.

Terms such as “typical,” “typically,” “common,” “commonly,” “often,” and the like are used to refer to elements or steps that frequently occur in the disclosure. Unless otherwise specified, these terms are not used to imply that any element or step is known in the prior art, much less that the element or step occurs frequently in the prior art.

The term “stamping” when used with reference to a culture device refers to the process of spreading an inoculant over a growth area of a culture device. A “stamping tool” is a tool for stamping. This is a known use of the term “stamping” in the relevant art: many stamping tools are known in the art. The term “stamping” with respect to a culture device is not used in the sense of using an implement, such as a stamp, to make a mark with ink or some other pigment, nor is “stamping tool” used with respect to culture devices to refer to a tool for making a mark on a culture device.

Terms such as “a” and “an” are often used for convenience, however, such terms are not meant to imply that only one item is being referred to, and instead allow for more than one unless one and only one is specified or is clear from the context.

Previously, the most common method of using a culture device involves manually uncovering the culture device, for example by lifting or removing a lid or flap, to expose a growth area and pipetting an inoculant that contains the sample to be tested on the growth area. These operations require dexterity and fine motor skills, and therefore could only be performed effectively by hand. Similarly; moving the culture device from one location to another also had to be performed by hand because machines were not sufficiently dexterous to move the culture device without the potential to cause damage to the culture device or to affect the outcome of the culture. Even instruments such as the INOBOT that may be able to perform some of these operations cannot perform multiple operations as precisely as a human because its effector assembly (i.e., the portion that engages with and moves the culture device, cover, etc.) is insufficiently dexterous.

Thus, in order to grow a culture using the technology available prior to this disclosure, a human operator would move a culture device from a culture device stack or other initial location to a location where it would be inoculated. The culture device was manually uncovered, such as by lifting or removing a cover or flap, to expose a growth area of the culture device. A pipet or similar hand-held tool was then used to manually place a sample, which is typically a liquid sample and often referred to as an inoculant, on the growth area. The cover was then replaced over the growth area. In some cases a hand-held stamping tool was used to manually spread the inoculant over the growth area. The culture device was then manually moved to an incubator, where it would incubate for a suitable time, before testing, such as colony detection or colony enumeration. This process is labor intensive and requires a high degree of expertise and careful manipulation by a human operator.

Thus, a problem to be solved is making a robotic system that is sufficiently mobile and dexterous that it can carry at least one, and preferably multiple, operations that previously required too much dexterity for a robot and had to be carried out manually; more particularly the operations are operations relating to processing of culture plates. A problem to be solved can involve making an effector assembly that is sufficiently agile to be able to perform tasks that require fine motor skill, such as uncovering culture plates, for example by lifting a flap or removing a cover, moving culture devices, covering culture plates, for example by replacing a flap or cover, and the like. Related problems involve finding a machine that can automate culture device processing, and preferably that can process more than one culture device simultaneously. Another problem involves finding a method that automates one or more of the steps required to process a culture device, such as the steps of uncovering a culture device, inoculating the culture device, covering the culture device, moving the culture device from one location to another, and stamping the culture device. A related problem involves simultaneous, automated processing of more than one culture device.

Briefly, solutions lie in the disclosed movable effector assembly, which can be associated with a culture device processing system. The movable effector assembly and, when employed, culture derive processing system enable automated processing of culture devices. The disclosed methods of processing culture devices are also solutions.

The movable effector assembly includes one or more effector arms that are capable of mechanically interacting with a culture device. The one or more effector arms include one or more rotating segments that enable at least one of the one or more effector arms to rotate into the position that is required to complete a variety of tasks that are required to process culture devices. For example, plurality of rotating segments of the one or more effector arms enable the one or more effector arms to move a culture device, uncover a cover from the culture device to expose a growth area, and replace the cover over the growth area. The configuration of the rotatable segments facilitates the effector arm and movable assembly performing these and other tasks as required for mechanical and automated processing of culture devices.

The one or more effector arms can each include one or more attachment members. The attachment members serve to secure, hold, or grip a culture device or a portion thereof, such as a cover, lid, or flap, a stamping device tool, or in some cases a label. Any suitable member capable of carrying out the required tasks can serve as an attachment member. Exemplary attachment members include one or more vacuum cups, which can optionally be in communication with a suction source, one or more clasping mechanisms, such as a clamp, vice, grip, mechanical fingers, one or more magnets, or the like.

In some cases, it can be advantageous to label a culture device. The attachment member can be capable of placing or affixing a label on the culture device. The attachment member can also be capable of receiving a label from a labeler, such as a label dispenser or label printer, and then placing or affixing the label on a culture device. The attachment member can retain the label for a time sufficient to transport the label from the labeler to culture device, and then place or affix the label on the culture device.

The culture device processing system includes at least one movable effector assembly, such as those described herein, that is movably coupled to a modular support to enable longitudinal movement of the movable effector assembly along the modular support. The various segments of the effector arm can also move around or about one or more axes. The culture device processing system can be configured so that the movable effector assembly can, by rotating segments about the various axis as described herein and by moving along the modular support, move a culture device from an input location to a channel.

Once in the channel, the culture device can be further operated on, for example, by the movable effector assembly. For example, the movable effector assembly can uncover a growth area of the culture device.

The channel can also include an inoculation station, in which case the movable effector assembly can move the culture device from an input location to the inoculation station. The culture processing device, and more particularly the moveable effector assembly, can be in communication with one or more inoculators. The one or more inoculators can be any suitable device for inoculating a culture device, particularly a growth area of the culture device. Examples include one or more pipette, micropipettes, swabs, dispensers, for example for dispensing a quantity, such as a pre-determined quantity of solid or liquid inoculant, and particularly liquid inoculant. Once the culture device is in the inoculation station, the inoculator can inoculate the culture device, typically the growth area of the culture device.

The channel can also have an output location, to which the movable effector assembly can move the culture device after inoculation. The movable effector assembly can replace the cover of the culture device in any suitable location of the channel, typically the inoculation station or the output location, but other locations are also possible. Most commonly, the cover is uncovered once before inoculation and replaced after inoculation, but it is also possible to uncover and replace the cover multiple times depending on the protocol being carried out.

It is possible for a culture processing device processing system to have multiple channels, in which case there will typically be one of each of the components of the culture device processing system, such as the movable effector assembly, for each channel. Thus, a culture processing device with a first and second channel will typically have a first movable effector assembly associated with the first channel and a second movable effector assembly associated with the second channel. When a third channel is present, then the culture processing device will typically have a third movable effector assembly associated with the third channel. Thus, when there are multiple channels and movable effector assemblies a culture device can be moved from a first input location to a first channel, or from a second input location to a second channel, and so on. Further, each channel will typically have all of the components as discussed herein so each channel and its associated movable effector assembly is typically capable of performing all of the steps needed to process a culture device without the need to use any of the components of another channel. Thus, typically each channel will have an input location, output location, etc., uniquely associated with the channel.

In a typical configuration of a multi-channel culture device processing system, a plurality of modular supports positioned adjacent to one another are each coupled to one movable effector assembly. This configuration provides significant flexibility because it allows the movable effector assemblies from each channel to work either separately or, more particularly, separately and simultaneously to process multiple culture plates at the same time, or together to process a single culture plate. The result is a significant increase in one or more of efficiency, speed, accuracy, and throughput, in culture device processing.

An inoculation system can comprise a plurality of culture processing systems. Such inoculation systems are typically configured so that the channels of the plurality of culture processing systems have parallel longitudinal directions. One or more of channels can share a single input location, which can be configured to retain a stack or cartridge or culture devices, or they can have distinct input locations. One or more of channels may share a single output location, which can be configured to retain a stack or cartridge or culture devices, or they can have distinct output locations.

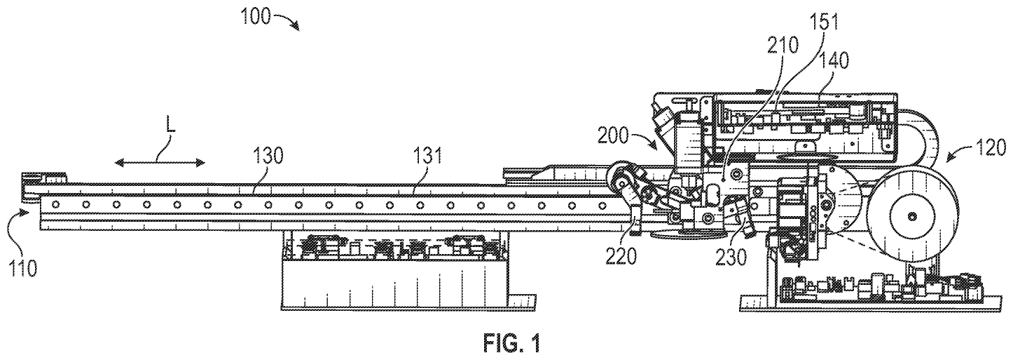

Turning to the Figures, which are not necessarily drawn to scale, and in some cases may be intentionally not drawn to scale in order to more clearly show various items, FIGS. 1-4 illustrate a culture device processing system 100 including a movable effector assembly 200. The culture device processing system 100 includes a first end 110 and a second end 120 opposite the first end 110. The culture device processing system 100 includes a modular support 130 that extends in a longitudinal direction L along the culture device processing system 100 from the first end 110 of the modular support 130 to a second end 120 of the modular support 130.

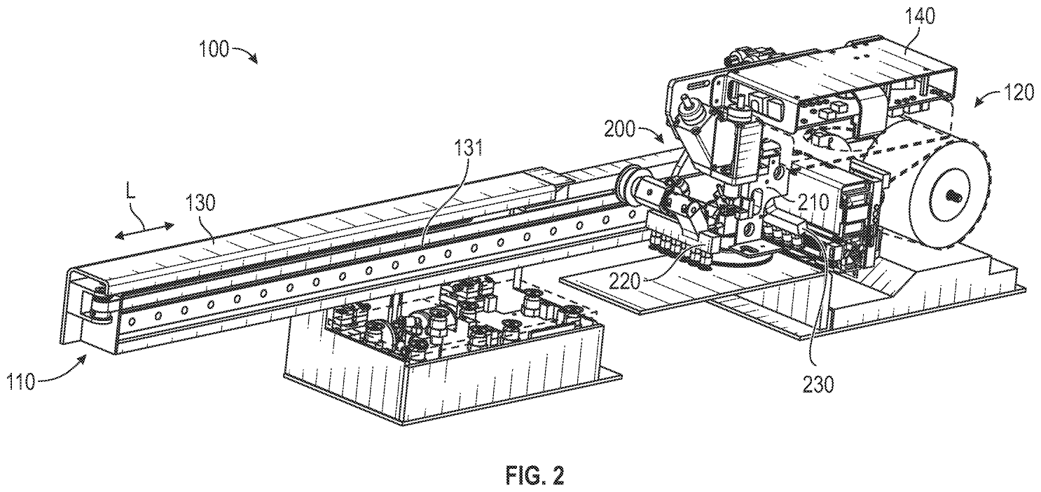

The culture device processing system 100 includes a housing 140 (FIGS. 1 and 2). The housing 140 may contain components, such as mechanical components, electrical components, or both mechanical and electrical components, for controlling the movable effector assembly 200. The culture device processing system 100 may be in communication with a computer (not shown). The computer or processor can be configured to execute a non-transient computer readable storage medium containing a set of computer operable instructions. When the instructions are executed by the computer in communication with the culture device processing system 100, the culture device processing system 100 executes operations to process the culture device, and particularly to perform one or more of the operations, steps, or methods described herein. The computer may control the culture device processing system 100 and the computer may have one or more software drivers for one or more controllers integrated with motors of the movable effector assembly 200. The computer may include multiple controllers for controlling movement of the movable effector assembly 200. The computer can be configured to send the necessary or desired automation commands to the movable effector assembly 200 for controlling the movable effector assembly 200 and one or more effector arms.

The movable effector assembly 200 includes a body 210 movably coupled to the modular support 130. The movable effector assembly 200 is configured to move in a longitudinal direction L along the modular support 130. The movable effector assembly 200 is shown in FIGS. 1-3 to be coupled to a track 131 of the modular support 130. In principle any components that can move an apparatus along a support or tack may be used for the movement of the movable effector assembly 200 along the modular support 130. Examples include an automated track, a rotating axel, actuated wheel systems and the like, any of which may be driven by an internal or external motor (not shown).

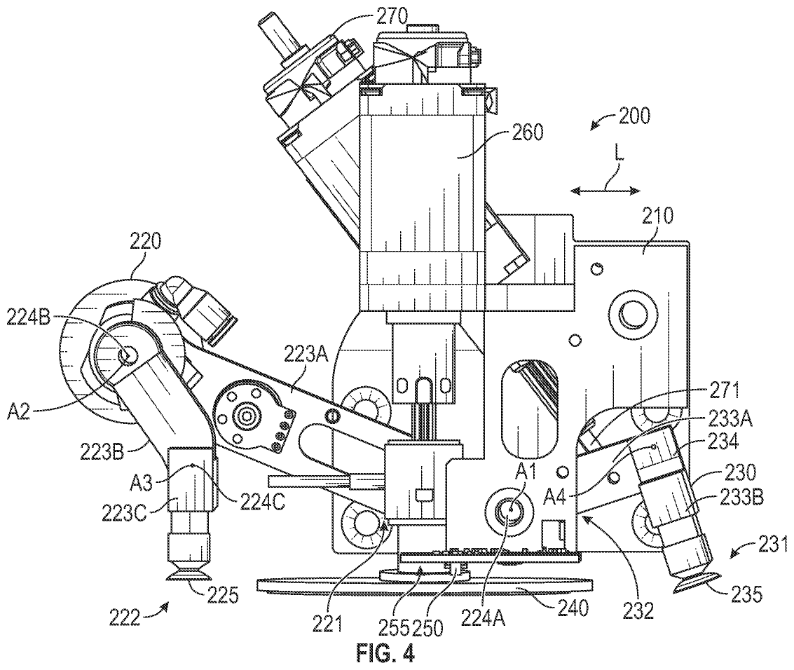

Movable effector assembly 200 includes one or more effector arms for coupling to a culture device and placing a label on the culture device. A first effector arm 220 extends out from the movable effector assembly body 210 in a first direction corresponding to the left side of FIG. 4. A second effector arm 230 may extend out from the body 210 of the movable effector assembly 200 in a second direction corresponding to the right side of FIG. 4. In FIG. 4, the second direction is substantially opposite the first direction. While this Figure shows a movable effector assembly 200 with two effector arms 220, 230, in other embodiments, the movable effector assembly 200 can have only one effector arm, or it can have three, four, or more effector arms.

A computer or processor 151, which can be internal or external, can be used to program the culture device processing system 100, including movable effector assembly 200.

FIG. 4 also shows details of first effector arm 220, which includes a first end 221 and a second end 222 opposite the first end 221. A first segment 223A of the first effector arm 220 is rotatably coupled to the body 210 at a first coupling 224A. The first coupling 224A is located proximate to the first end 221 of the first effector arm 220. The first segment 223A is rotatable, optionally by at least 180 degrees and particularly by 360 degrees, around a first axis of rotation A1. A second segment 223B is rotatably coupled to the first segment 223A at a second coupling 224B. The second segment 223B is rotatable, optionally by at least 180 degrees, and particularly by 360 degrees, around a second axis of rotation A2. As depicted in this Figure, the first effector arm 220 includes a third segment 223C rotatably coupled to the second segment 223B at a third coupling 224C. The third segment 223C may be rotatable, optionally by at least 180 degrees, and particularly by 360 degrees, around a third axis of rotation A3. Alternatively, the third segment 223C may be statically coupled to the second segment 223B. In some embodiments the third segment 223C may be omitted and only the first segment 223A and second segment 223B are present. In some embodiments, additional segments, which can be rotatably or statically coupled to adjoining segments, can be included

A first attachment member 225 is located at the second end 222 of the first effector arm 220. The first attachment member 225 is for removably coupling the first effector arm 220 to at least one culture device (not shown) placed upon a channel (not shown) of the culture device processing system. In addition, it can be for receiving a label from a labeler (neither shown here), for placing a labeler on a culture device, or both.

Each of the first, second and third axes of rotation A1, A2, A3 are orthogonal to the page bearing the Figures, and so are represented in these Figures by points of rotation that extend through the page so that each of the three axes as extending perpendicular to the longitudinal direction L. Thus, each of the axes of rotation A1, A2, A3 are perpendicular and non-colinear.

Note that if the movable effector assembly 200 is rotatable with respect to direction L, the axes can also move so that they are not perpendicular to direction L. The particular representation in the Figures is thus for clarity and does not require the axes A1, A2, A3 to be perpendicular to direction L.

The first effector arm 220 can be capable of rotating 360 degrees around the first axis of rotation A1. The first, second, and third segments 223A, 223B, 223C are individually rotatable along the parallel, non-colinear axes first, second, and third rotatable axes A1, A2, A3. This provides the first effector arm 220 sufficient dexterity to replicate manual operations in culture processing so that the movable effector assembly 200 is capable of positioning the first attachment member 225 in a desired position to couple to and move the culture device or a cover on a culture device (neither shown in this Figure), for example, as part of performing the tasks described herein, without producing any bubbles or distortions in the culture device that may compromise the inoculation process or may frustrate detection or enumeration of colonies on the culture plate after processing.

Particular tasks that can be performed include moving a culture device from one location for another. For example, from an input location to a channel, such as to an inoculation station, and from a channel, such as an inoculation station, to an output location. Additionally, the independent rotation of the first, second, and third rotatable segments 223A, 223B, 223C can enable the first effector arm 220 to uncover and cover the culture device (not shown) with a cover (not shown) such that an inoculant may be added to the growth area.

More particularly, the culture device can have a cover that is a thin, flexible sheet. In such cases the cover is usually attached to the culture device at a first end of the cover to form a hinge-like construction. In these more particular cases, the effector arm is capable of grasping, and the method includes the step of grasping, the cover at a second end of the cover that is opposite the first end. Most particularly, the effector arm is capable of rolling, and the method includes the step of rolling, the thin, flexible cover sheet back towards the first end of cover sheet without separating the cover sheet from the culture device or lifting the culture device off of the channel.

The types of culture devices that can be employed, however, are not limited to those with thin, flexible cover sheets. Culture devices with stiff glass or plastic cover sheets, such as Petri dishes, can also be employed and the movable effector assembly and culture device processing system described herein can be configured to operate on them.

The second effector arm 230 is depicted as including components that generally correspond in form and function to the components of the first effector arm 222. However, it is possible for the first and second effector arms 220, 230, to be configured differently. Here, the second effector arm 230 includes a first end 231 and a second end 232 opposite the first end 231. The second effector arm 230 additionally includes a first segment 233A and a second segment 233B. The first segment 233A is rotatably coupled to the body 210. The first segment 233A may be coupled to the body 210 at the first coupling 224A and rotatable around the first axis of rotation A1. Alternatively, the first segment 233A of the second effector arm 230 may be coupled to the body 210 at a position that is not colinear with the first axis of rotation A1, such that the first and second effector arms 220, 230 are coupled to the body 210 at separate positions. The second segment 233B of the second effector arm 230 is rotatably coupled to the first segment 233A at a fourth coupling 234. The second segment 233B of the second effector arm 230 may be rotatable around a fourth axis of rotation A4. In FIG. 4, the second segment 233B of the second effector arm 230 is rotatable to a third position such that the second effector arm 230 is positioned straight in the longitudinal direction L towards the second end of the culture device processing system 100 in order for the second attachment member to take or receive a label from the label holder (neither shown in this Figure.)

A second attachment member 235 is located at the second end 232 of the second effector arm 230. The second attachment member 235 may be configured to receive and retain a label from a labeler (not shown) and place the label on the culture device.

In any embodiment, the first and second attachment members 225, 235 may include one or more vacuum cups or an array of more than one vacuum cups, which vacuum cup or cups can optionally be in communication with a suction source, one or more clasping mechanisms, such as a clamp, vice, grip, mechanical fingers, one or more magnets, or the like (for clarity, this level of detail is not shown in the Figure).

The movable effector assembly 200 can contain a stamping tool 240, that in FIG. 4. is removably coupled to the body 210. The stamping tool 240 may be coupled to the movable effector assembly 200 via a coupling mechanism 250 near a base 255 of the movable effector assembly 200. Various coupling mechanisms may include magnetic coupling, vacuum suction coupling, mechanical coupling, such as by a grip, vice, friction fit, or the like, or any other coupling mechanism. The coupling mechanism 250 is configured to retain the stamping tool 240 until, for example, it is to be removed for cleaning or replacement. When the stamping tool 240 is removed from the coupling mechanism 250 a second stamping tool that corresponds to the type of culture device being employed can replace the first stamping tool 240. Different stamping tools may be compatible with different culture devices, and each stamping tool 240 is configured to provide a consistent stamping process. The stamping process provides for spreading inoculant within the growth area of a culture device (not shown in FIG. 4), and particularly uniformly within the growth area of a culture device, without causing the inoculant to spill or otherwise escape from the growth area.

The movable effector assembly 200 can also include a first actuator 260 and a second actuator 270 coupled to the body 210. Activation of the first actuator 260 enables the stamping tool 240 to move between a first position wherein the stamping tool 240 is not in contact with a culture device (not shown), and a second position wherein the stamping tool 240 is in contact with a culture device. Activation of the second actuator 270 enables movement of the second effector arm 230 between a first position wherein the second attachment member 235 is not in contact with a culture device, and a second position wherein the second attachment member 235 is in contact with the culture device for placing a label on the culture device. The second actuator 270 is coupled to the second effector arm 230 by a shaft 271 that is positioned up or down depending on whether the second effector arm 230 is in the first or second position. However, it is also possible that the second effector arm 230 can be actuated at the first coupling 224A to rotate from the first position to the second position.

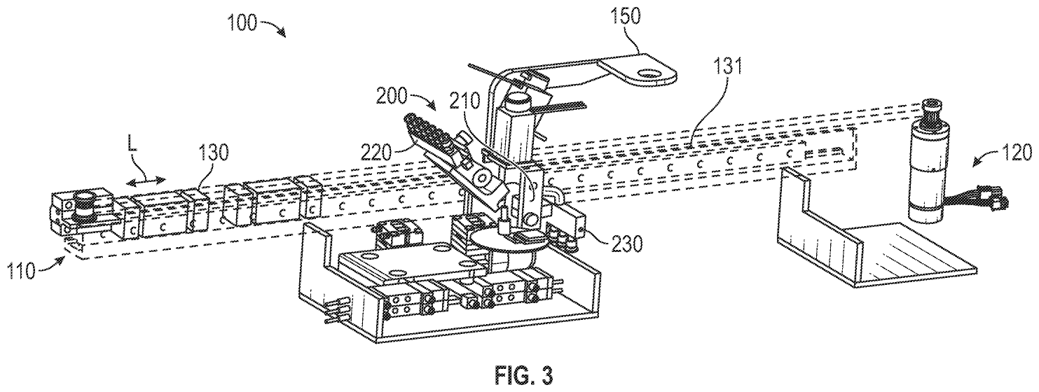

Returning to FIG. 3, an image capture device 150 is coupled to the body 210 of the movable effector assembly 200. The image capture device 150 is for capturing an image of the culture device placed on the channel 400. The image capture device 150 or an internal or external computer or processer 151 (see FIG. 1) can retrieve an image of one or more retrieved indicia, typically that relate to the expected type of culture device, for example a culture device corresponding to a particular type of microorganism, a culture device having particular characteristics, a culture device bearing a particular type of indicia (bar code, QR code, writing, etc.), a culture device having particular dimensions, or the like, and compare the captured indicia of the culture device with the received indicia. Most commonly, the captured indicia is an image or part of an image of the culture device, such as a bar code, QR code, color, text, or the like, that is marked on the culture device. Most commonly, the received indicia is an image or part of an image of the culture device, such as a bar code, QR code, color, text, or the like, that would be marked on the expected type of culture device.

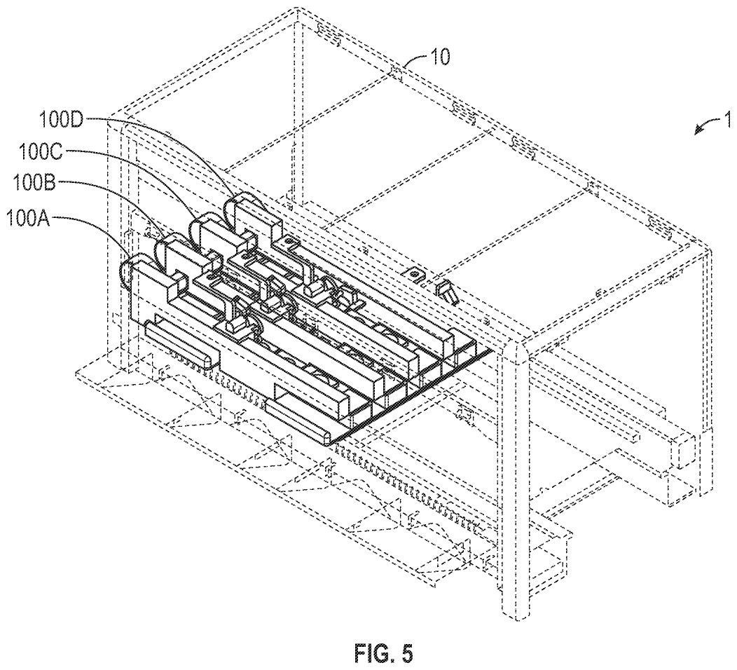

Referring to FIG. 5, an inoculation system 1 can include a plurality of culture device processing system (100A, 100B, 100C, 100D). In FIG. 5 the plurality of culture processing systems are adjacent and have parallel longitudinal directions, but other arrangements are also possible. The inoculation system includes a housing 10 that accommodates the plurality of culture device processing systems. Each of the plurality of culture device processing systems may be configured for processing the same or different type or types of culture. This arrangement can further enhance the efficiency, throughput, and speed of processing a large number of culture devices.

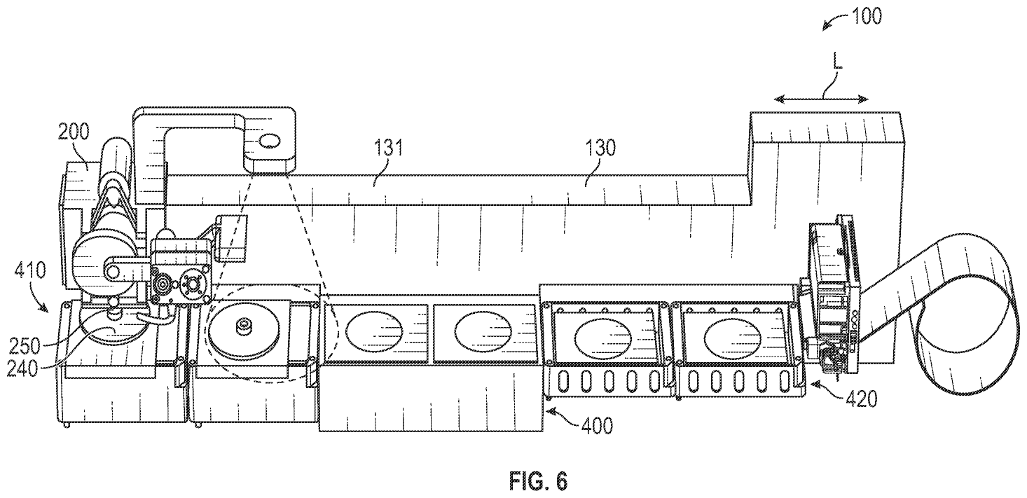

FIGS. 6-16 illustrate particular ways of operating a culture device processing system 100. FIG. 6 illustrates a step of coupling the movable effector assembly 200 to a stamping tool 240 that is suitable for use with the type of culture device 500 being employed. The coupling mechanism 250 of the movable effector assembly 200 securely couples the stamping tool 240 to the movable effector assembly 200. The movable effector assembly 200 can move in the longitudinal direction L along the modular support 130 in order to position the coupling mechanism 250 in a location for coupling to the stamping tool 240.

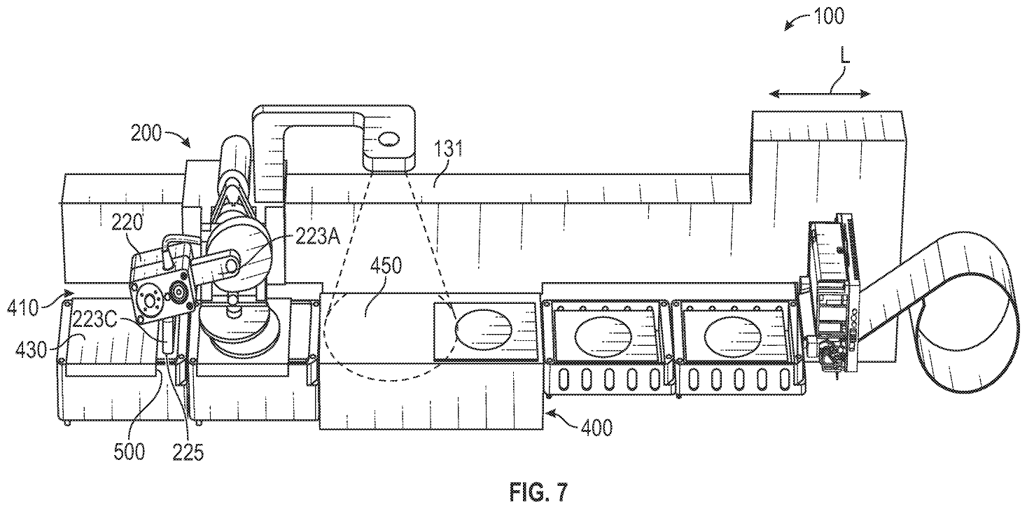

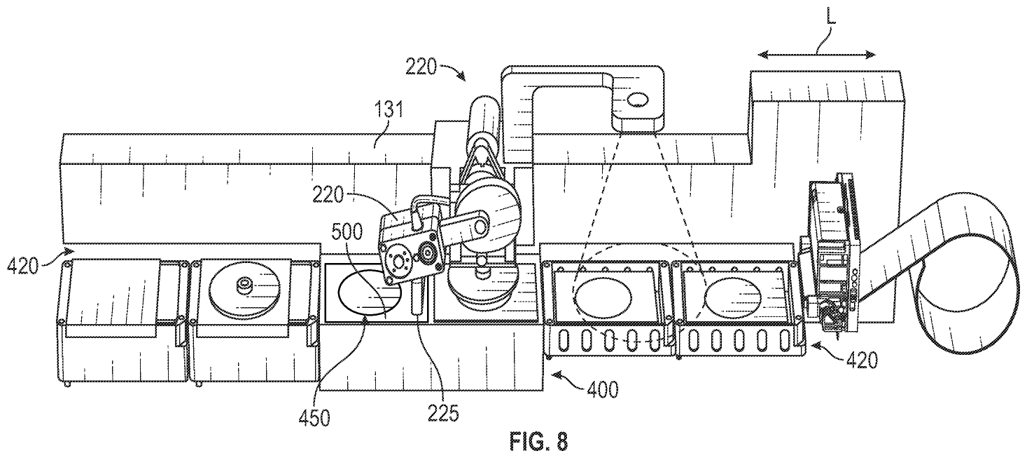

FIG. 7 illustrates the first effector arm 220 of the movable effector assembly 200 coupling the first attachment member 225 to the culture device 500 at the input location 430 of the channel 400. The input location 430 may include a single culture device or stack of multiple culture devices. The movable effector assembly 200 is actuated towards the first end 410 of the channel 400 to enable the first attachment member 225 to engage the culture device 500. The first effector arm 220 is shown in an L-like position, with the first segment 223A oriented in the longitudinal direction L towards the first end of the channel 400; however, depending on the particular configuration and dimensions of the first effector arm 220 and the other components of the culture processing system, other orientations are also possible. At least the third segment 223C may rotate perpendicular to the longitudinal direction in order to engage the channel 400. The first attachment member 225 engages and retains the culture device 500 for transfer to a separate location on the channel 400. Once the culture device 500 is securely coupled to the first attachment member 225, the movable effector assembly 200 may move to a position for placing the culture device 500 on a first position of the channel 400 (FIG. 8).

FIG. 8 illustrates the first effector arm 220 placing the culture device 500 on the first position 450 of the channel 400. The movable effector assembly 200 has moved from the position that it was in in FIG. 7 along the longitudinal direction L towards the second end 420 of the channel 400, and in this FIG. 8 is in an appropriate location to position the retained culture device 500 at the first position 450 of the channel 400. The first attachment member 225 is oriented to ensure that the culture device 500 is accurately placed on the first position 450 of the channel 400. Once the culture device is in the first position 450, the first attachment member 225 can disengage from the culture device. 500

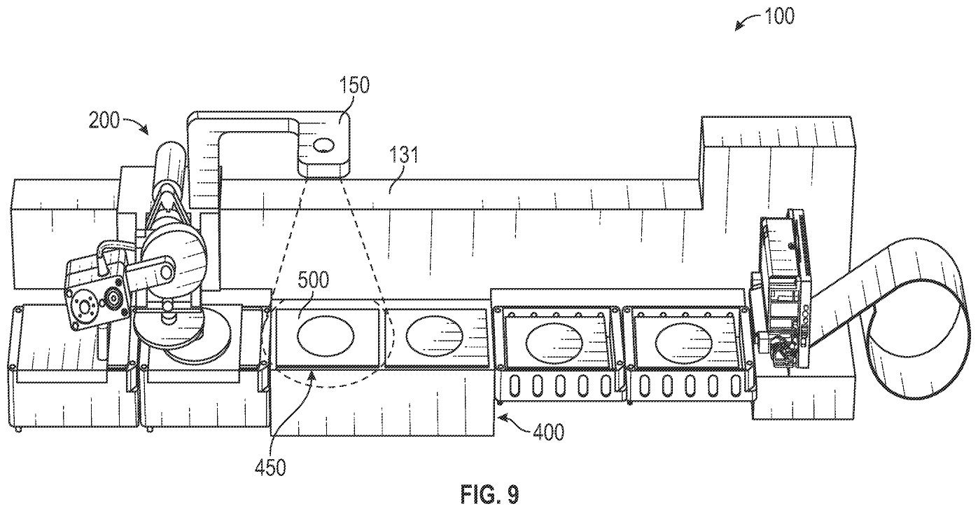

FIG. 9 illustrates the image capture device 150 capturing an image of the culture device 500. The image capture device can capture captured indicia, which is typically an image or part of an image of the culture device 500. The captured indicia can be, for example, a bar code, QR code, color, text, or the like, that would be marked on the expected type of culture device culture device, but can also be a shape, dimensions, or the like, of the culture device 500, or a property of the inoculant in the growth area, such as the color, presence of bubbles, or the like.

The captured indicia can be used as a quality assurance measure to assist in avoiding error. In use, a user can provide, to a computer or processer in communication with the image capture device 150, and which may be internal or external to the image capture device 150 and the culture plate processing system 200, a worklist that includes information regarding the number and type of plates to be processed, and the like. This worklist, when employed, provides a list of expected culture devices, which represents for example the type of culture devices that are to be processed and order in which they are to be processed. A non-transient computer-readable storage medium can be configured, typically pre-configured, to store retrieved indicia relating to various types of possible expected culture devices. The retrieved indicia is typically an image or part of an image of the expected culture device, such as a bar code, QR code, color, text, or the like, that would be marked on the expected type of culture device culture device, but the received image could also relate to the dimensions, of the culture device, the shape of the culture device or the like.

The image capture device 150 or an internal or external computer or processer can retrieve, from the non-transient computer-readable storage medium, the retrieved indicia of an expected type of culture device. The processor can compare the captured indicia with the retrieved indicia to determine whether the culture device 500 that is being processed is of the same type as the expected type of culture device. The computer or processer can be further configured to provide a warning if the expected type of culture device does not match the type of culture device 500 that is being processed. The image capture device 150, along with the processer (if employed) can function as a checkpoint to ensure that the culture device processing system is operating on the expected type of culture device, thereby correcting user error, for example, an error in placing the wrong type of culture device for the inoculant being employed on the input location for processing.

Additional or in the alternative, the captured indicia can be compared to the retrieved indicia to determine the type of culture device 500 from which the captured indicia were captured. The non-transient computer-readable storage medium can contain a plurality of sets of instructions, which can be for example details regarding how to process different types of culture devices, that correspond to different types of culture devices. The processer can then retrieve a set of instructions that corresponds to the type of culture device from which the captured indicia was captured.

Additionally or in the alternative, the image capture device 150 may detect the position of the culture device 500 to ensure that the culture device is correctly positioned on the first position 450 on the channel 400.

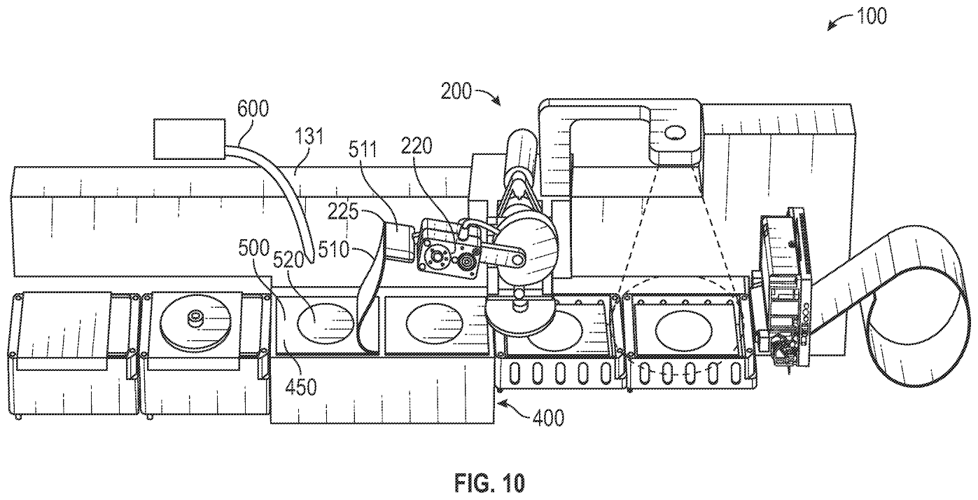

FIG. 10 illustrates the first effector arm 220 removing the cover 510 of the culture device 500 to expose a growth area 520 on the culture device 500. The first attachment member 225 is lowered to a first position in communication with the channel 400 wherein the first attachment member 225 engages and secures a first end 511 of the cover 510. The first attachment member 225 is subsequently moved to a second position away from the channel 400 to lift the flap and expose the growth area 520 of the culture device 500. Once the growth area 510 is exposed an inoculant may be added to the growth area 520 of the culture device 510.

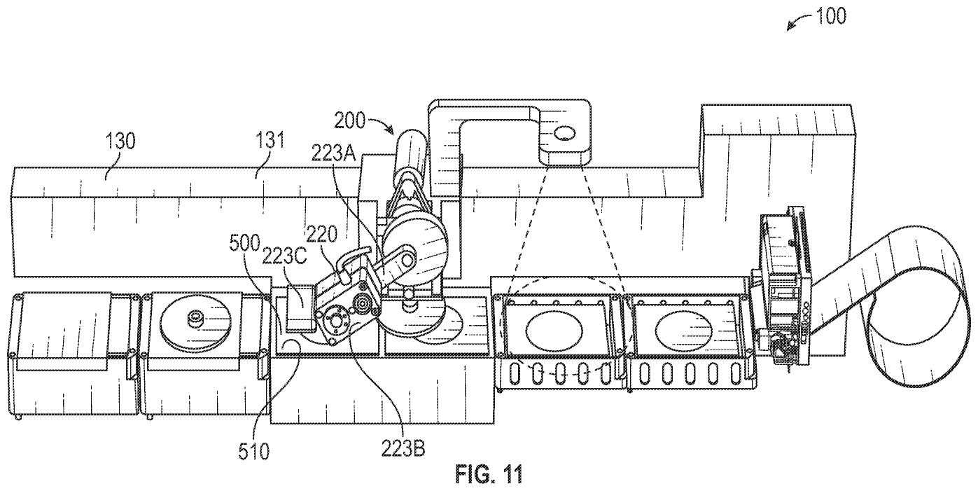

The inoculant may be added by an inoculator 600, which is usually a pipetting apparatus and more particularly an automated pipetting apparatus. In some cases, the inoculant is added manually but this is less common. Although it is preferred that the culture device processing system 100 includes inoculator 600, such as one or more pipette, micropipettes, swabs, dispensers, for example for dispensing a quantity, such as a pre-determined quantity of solid or liquid inoculant, particularly a pipette (not shown) that delivers a the inoculant. Particular inoculators 600 are automated, and more particularly an automated pipetting apparatus which can be a micropipetting apparatus or other pipetting apparatus depending on the volumes of inoculant to be used, which can deliver inoculant onto the growth area 520. The inoculator 600 is particularly in communication with the moveable effector assembly 200 to inoculate the culture device 500, usually when it is located at first position 450 on the channel, which in this Figure also serves as an inoculation station. Notably, it is not required that inoculator 600 be present, and several figures, for example FIG. 11, depict culture device processing system 100 with no inoculator 600.

FIG. 11 illustrates the first effector arm 220 replacing the cover 510 over the growth area 520 once the inoculant (not shown) has been added. Rotation of the first, second, and third segments 223A, 223B, 223C ensures that the cover 510 is properly replaced, for example to effectively seal growth area 520, and that the inoculant within the growth area 520 is not disturbed.

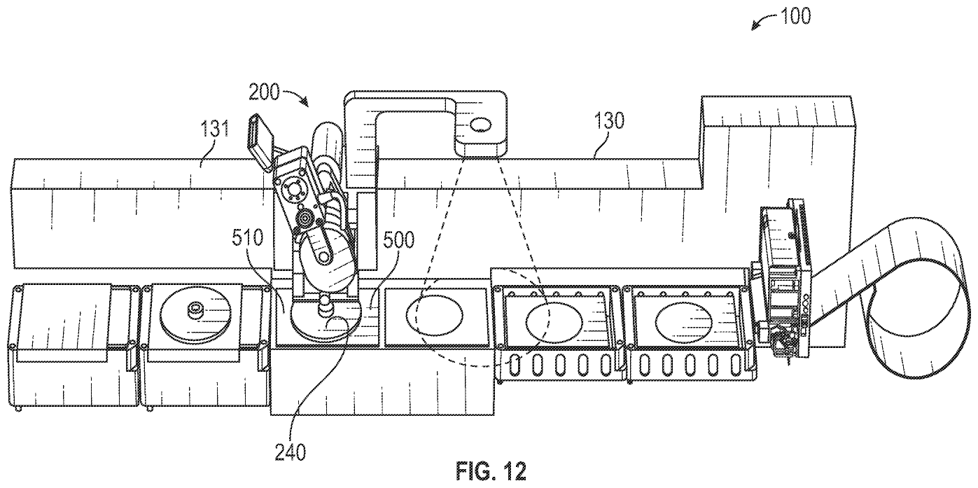

FIG. 12 illustrates the optional step of stamping of the culture device 500. This step may not be necessary depending on, for example the type of culture plate, inoculant, and inoculator employed, and the requirement of any detection or enumeration that may later be performed on the culture device. In FIG. 12, the cover 510 of the culture device 500 has already been replaced over the growth area 520 of the culture device 500, and the movable effector assembly 200 is moved along the modular support 130 to position the stamping tool 240 over the culture device 500. The first actuator (not shown) moves the stamping tool 240 from a first position, wherein the stamping tool 240 is disengaged from the culture device, to a second position wherein the stamping tool 240 contacts the culture device 500. The stamping tool 240 contacts the cover (not show) of the culture device 500 to apply pressure to the growth area (not shown) and spread the inoculant (not shown) within the growth area. The stamping tool 240 is returned to the first position.

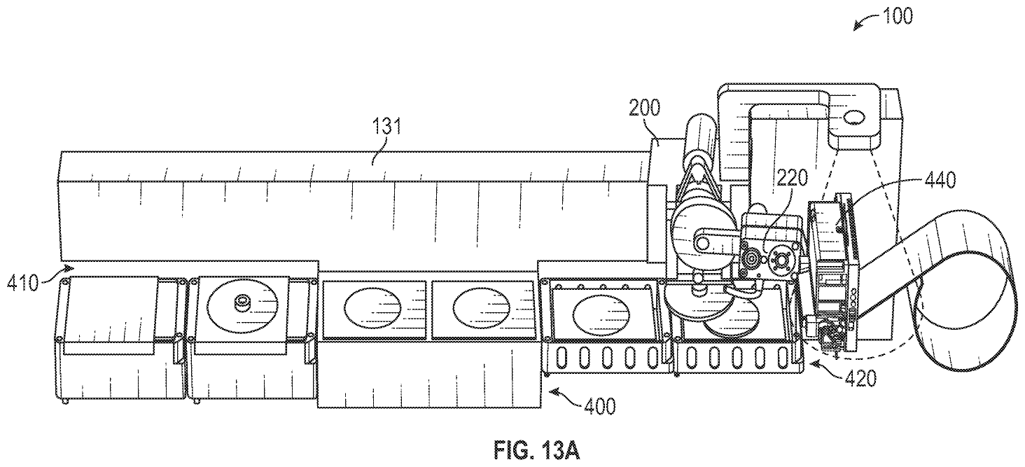

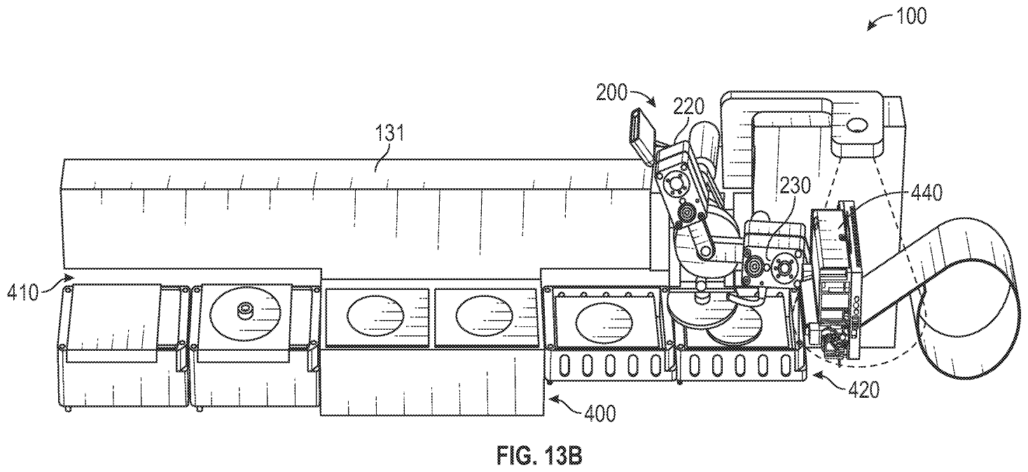

FIG. 13A shows the first effector arm 220 receiving a label from the labeler 440 at the second end of the channel 400. The first effector arm 220 may rotate from a side facing the first end 410 of the channel 400 to a side facing the second end 420 of the channel 400 of the movable effector assembly 200 to engage the labeler 440. The first attachment member (not shown) takes the label from the labeler 440. The label can, for example, be printed with information related to the culture device (not shown) being processed. During this operation, the first attachment member serves as label holder to hold or retain the label until the label is placed, for example, on the culture device (not shown)

FIG. 13B illustrates an alternative to the embodiment illustrated in FIG. 13A. In FIG. 13B, movable effector assembly 200 has two effector arms: first effector arm 220 and second effector arm 230. Here, the second effector arm 230 takes the label from the labeler 440. The second attachment member (not shown) of the second effector arm 230 engages the labeler 440 and takes a label. The label can, for example, be printed with information related to the culture device (not shown) being processed. The first effector arm 220 is positioned so as not to interfere with or contact the labeler 440 or the second effector arm 230 or any culture devices (not shown) that may also be present within channel 400.

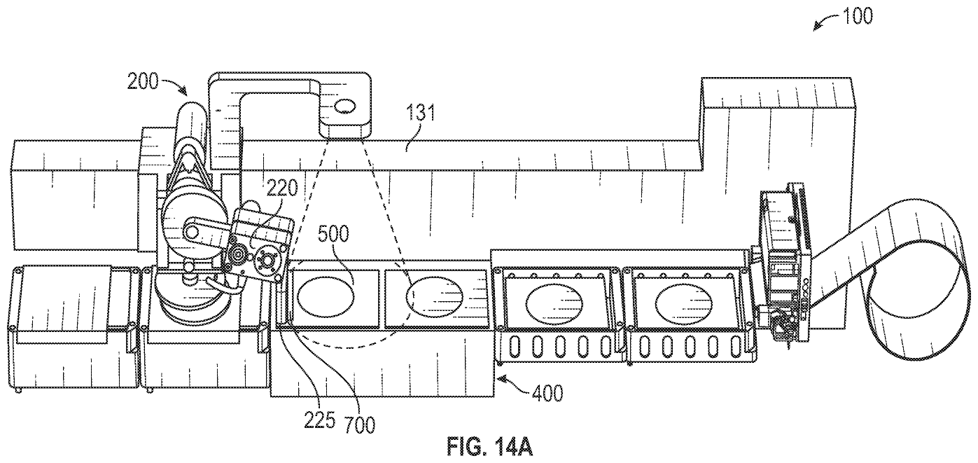

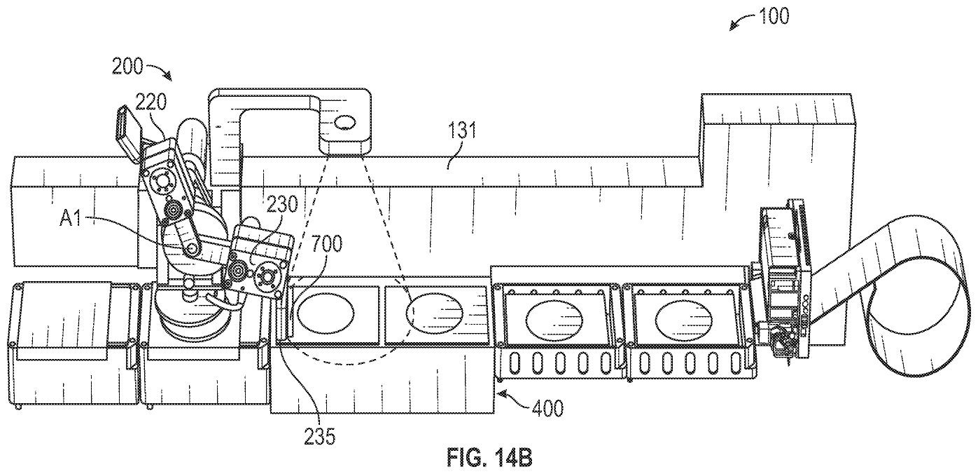

FIG. 14A illustrates shows the first effector arm 220 applying the label (not visible in this Figure) to the culture device 500. The first attachment member 225, which acts as a label holder, is positioned towards the channel 400 to engage the culture device 500 and apply the label to the culture device 500.

FIG. 14B shows a movable effector assembly 200 having two effector arms: first effector arm 220 and second effector arm 230. In FIG. 14B, second effector arm 230 aligns with the culture device (not visible in this Figure) and the second attachment member engages the culture device to apply the label (not visible in this Figure) to the culture device.

A culture processing system can have a single effector arm, for example as shown in FIGS. 13A and 14A, or multiple moveable effector arms, for example as shown as in FIGS. 13B and 14B. When there are multiple effector arms, the culture device can be configured so that each of the multiple moveable effector arm are each capable of independently accomplishing the steps in the processing of culture devices. Employing only one single effector arm saves space and avoid added components that may be cumbersome when processing a culture device.

Alternatively, multiple effectors arms may be configured to perform different steps in the processing of culture devices. This configuration can provide increased efficiency by including an additional arm for the labeling step as opposed to requiring full rotation of the first effector arm 220 during the labeling step. Thus, either embodiment might be implemented depending on the priorities and needs of the user.

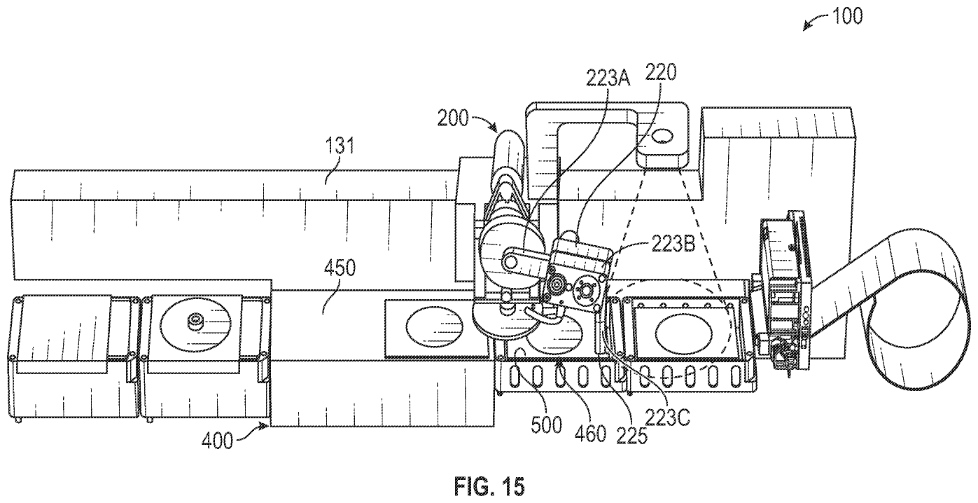

FIG. 15 illustrates the first effector arm 220 coupled to the culture device 500 and placing the culture device 500 at an output location 460. The first second and third segments 223A, 223B, 223C are rotatable about their respective axes of rotation to place culture device 500 at output location 460. The image capture device 150 can verify the correct position of the culture device 500 once the device is placed at the output location 460. Once correct positioning of culture device 500 is confirmed, the first attachment member 225 can disengage from the culture device 500 at the output location, after which time the culture device processing system 100 can begin processing another culture device.

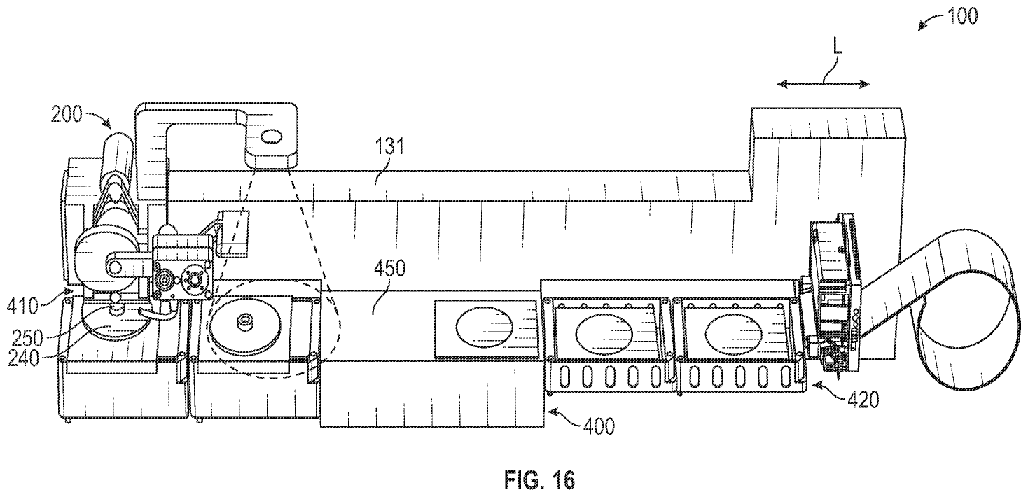

FIG. 16 illustrates the step of returning the movable effector assembly 200, which here is still connected to stamping tool 240, to first end 410 of the channel 400. In some cases, a washing station (not shown) is present and the stamping tool 240 is placed in the washing station. Typically stamping tool 240 is reusable and while it can be replaced or washed when worn or soiled, replacement is usually not necessary after each use. If the stamping tool 240 is discarded, the coupling mechanism 250 can couple to a new stamping tool (not shown) for continued processing of culture devices.

In FIGS. 15 and 16, first position 450 is empty because the moveable effector assembly 200 has not yet placed a new culture device to be processed in that location.

The culture device processing system 100 is adapted to include multiple stamping tools that may be identical or may vary in function, shape, or size. These stamping tools may be positioned along the support in a location accessible to the coupling mechanism 250 of the movable effector assembly 200. Thus, the stamping tool most suitable for use with the culture device being processed can be selected.

Any of the culture device processing systems disclosed herein can store one or a plurality of processed culture device, unprocessed culture devices, or both in a storage system, for example as a stack or in a cartridge. The storage system will typically be located at the input location for unprocessed culture devices, at output location for inoculated (processed) culture devices, or both, but other locations are possible. The culture device processing systems, when a plurality of plates is present, can remove each or a plurality of culture devices from the storage system and return each or a plurality of culture devices to the storage system (either the same storage system or a different storage system). Culture processing systems as described herein even be configured to use movable effector assemblies to place culture devices in an incubator, for example, after the storage plates have been inoculated or at other appropriate times.

1. A culture device processing system, comprising:
a first input location for retaining at least one culture device,
a first channel comprising at least a first position for receiving a culture device and retaining a received cultured device,
a movable effector assembly in communication with the channel, the movable effector assembly comprising one or more effector arms,
the one or more effector arms comprising a first effector arm having a first attachment member for removably coupling the first effector arm to the at least one culture device, and
wherein the first effector arm is for:
moving the at least one culture device from the first input location to the first position of the first channel, and
uncovering a growth area of the at least one culture device.

2. The culture device processing system of claim 1, wherein the first effector arm is also for:
covering the growth area with a cover; and
moving the at least one culture device to an output location

3. The culture device processing system of claim 1, wherein the first effector arm comprises an attachment member for placing a label on the culture device.

4. The culture device processing system of claim 1, wherein the one or more effector arms further comprises a second effector arm.

5. (canceled)

6. The culture device processing system claim 1, further comprising:
a second channel,
a second input location associated with the second channel, and
a second movable effector assembly in communication with the second channel, the second movable effector assembly comprising one or more effector arms.

7. The culture device processing system of claim 1, wherein at least one of the one or more effector arms comprises a first segment that is rotatable by at least 180 degrees, and optionally by 360 degrees, around a first axis of rotation.

8. The culture device processing system of claim 1, wherein the second effector arm comprises a first segment that is rotatable, optionally by at least 180 degrees and further optionally by 360 degrees, around the first axis of rotation.

9. The culture device processing system of claim 1, wherein the first effector arm comprises a second segment that is rotatable, optionally by at least 180 degrees and further optionally by 360 degrees, around a second axis of rotation that is parallel with the first axis of rotation and that is not colinear with the first axis of rotation.

10-11. (canceled)

12. The culture device processing system of claim 1, wherein the first effector arm is configurable between a first position wherein the first attachment member is not coupled to the culture device, and a second position wherein the first attachment member is removably coupled to the culture device.

13-29. (canceled)

30. A method of operating an effector assembly, comprising:
causing at least one effector arm of a movable effector assembly to place a culture device onto a first area of a channel;
causing a first segment of the at least one effector arm to uncover a cover of the culture device to expose a growth area of the culture device;
adding an inoculant to the growth area of the culture device;
causing the at least one effector arm to replace the cover over the growth area;
causing the at least one effector arm to move the culture device to an output location.

31. The method of claim 30, further comprising a step of stamping culture device with a stamping tool coupled to the movable effector assembly.

32. The method of claim 30, wherein the step of causing the at least one effector arm to place the culture device onto a first area of the channel further comprises rotating a second segment of the at least one segment of the effector arm around a first axis of rotation.

33. The method of claim 30, wherein the step of causing the at least one effector arm to place the culture device onto a first area of the channel further comprises rotating the at least one effector arm around a second axis of rotation that is not colinear with the first axis of rotation and that is optionally parallel to the first axis of rotation.

34. The method of claim 30, further comprising:
a step of simultaneously rotating the first segment of the at least one effector arm around the first axis of rotation and the second segment of the at least one effector arm about the second axis of rotation.

35. The method of claim 30, further comprising:
a step of rotating a third segment of the at least one effector arm around a third axis of rotation that is not colinear with the first axis of rotation or the second axis of rotation, and that is optionally parallel with the first axis of rotation and the second axis of rotation.

36. The method of claim 30, further comprising:
a step of simultaneously rotating at least two of the first, second, and third segments of at least one effector arm around at least two of the first, second, and third axes of rotation.

37. The method of claim 30, wherein further comprising moving the movable effector assembly in a longitudinal direction along a track, optionally simultaneously with rotating at least one of the first, second, and third segments of at least one effector arm around at least two of the first, second, and third axes of rotation, and further optionally simultaneously with rotating at least two of the first, second, and third segments of at least one effector arm around at least two of the first, second, and third axes of rotation.

38. The method of claim 30, further comprising:
a step of rotating the at least one effector arm about one or more of the first axis of rotation, the second axis of rotation, and the third axis of rotation to cause the at least one effector arm to apply a label to the culture device.

39. (canceled)

40. The method of claim 30, further comprising:
a step of capturing an image of the culture device with an image capture device.

41-44. (canceled)

45. A method comprising causing a processor in communication with a culture processing system of effector assembly to execute a non-transient computer readable storage medium containing a set of computer operable instructions, that when executed by the computer in communication with the movable effector assembly, causes the movable effector assembly to carry out the method of claim 30.

46. The method of claim 30, wherein the movable effector assembly comprises:
a body movably coupled to a modular support;
one or more effector arms coupled to the body, comprising:
a first end and a second end opposite the first end;
a first segment rotatably coupled to the body at the first end of the one or more effector arms,
a second segment rotatably coupled to the first segment;
and a first attachment member at the second end of the one or more effector arms;
wherein the first segment is rotatable around a first axis of rotation, optionally by at least 180 degrees and further optionally by 360 degrees,
the second segment is rotatable around a second axis, optionally by at least 180 degrees and further optionally by 360 degrees; and
wherein the second axis of rotation is not colinear with the first axis of rotation.

47. (canceled)
